(19) United States
(12) Patent Application Publication
(10) Pub. No.: US 2026/0061215 A1
(43) Pub. Date: Mar. 5, 2026

(54) APPARATUS FOR INDUCING A NUCLEAR MAGNETIC RESONANCE

(71) Applicant: Robert Allen Wilson, Oak Island, NC (US)

(72) Inventor: Robert Allen Wilson, Oak Island, NC (US)

(21) Appl. No.: 19/315,171

(22) Filed: Aug. 29, 2025

Related U.S. Application Data
(60) Provisional application No. 63/688,731, filed on Aug. 29, 2024.

(51) Int. Cl. A61N 2/02; A61N 2/00

(52) U.S. Cl. CPC A61N 2/02; A61N 2/002

(57) ABSTRACT

The present disclosure provides an apparatus for inducing a nuclear magnetic resonance. Further, the apparatus may include a magnetic unit which may be configured for generating a static magnetic field. Further, the static magnetic field may be characterized by a magnetic field characteristic. Further, the apparatus may include a radiofrequency coil unit which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the apparatus may include a controlling unit communicatively coupled with each of the magnetic unit and the radiofrequency coil unit. Further, the controlling unit may be configured for controlling one or more of the magnetic field characteristic and the radiofrequency characteristic to create a therapeutic effect in the user's body.

### FIELD OF DISCLOSURE

The present disclosure generally relates to the field of medical and laboratory equipment. More specifically, the present disclosure relates to an apparatus for inducing a nuclear magnetic resonance.

### BACKGROUND

The present disclosure relates generally to the field of medical technology. The field of medical technology directly impacts the accessibility, efficiency, and precision of healthcare delivery across a wide range of clinical and non-clinical environments. The ability to deliver therapeutic effects without reliance on traditional pharmaceutical logistics—such as pills, injections, or infusions—may offer significant improvements in global health outcomes, especially in underserved, remote, or resource-constrained settings.

Existing systems and methods for drug delivery rely heavily on manufacturing, storage, transport, and administration of chemical formulations, which introduces various challenges. The challenges include the risks of contamination, degradation, and errors in dosage or administration. Furthermore, the existing systems may require substantial healthcare infrastructure, trained personnel, and continuous supply chain operations.

In addition, conventional therapeutic methods often lack real-time adaptability to patient-specific responses and are typically constrained in environments such as space missions, isolated communities, or emergency response scenarios. Security concerns, such as ensuring that only the intended recipient receives the therapeutic effect, further complicate the deployment of such systems in public or communal settings.

Therefore, there is a need for an improved apparatus for inducing a nuclear magnetic resonance that can overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides an apparatus for inducing a nuclear magnetic resonance. Further, the apparatus may include a magnetic unit which may be configured for generating a static magnetic field. Further, the static magnetic field may be characterized by a magnetic field characteristic. Further, the apparatus may include a radiofrequency coil unit which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the apparatus may include a controlling unit communicatively coupled with each of the magnetic unit and the radiofrequency coil unit. Further, the controlling unit may be configured for controlling one or more of the magnetic field characteristic and the radiofrequency characteristic to create a therapeutic effect in the user's body.

The present disclosure provides an apparatus for inducing a nuclear magnetic resonance. Further, the apparatus may include a magnetic unit which may be configured for generating a static magnetic field. Further, the static magnetic field may be characterized by a magnetic field characteristic. Further, the apparatus may include a radiofrequency coil unit which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the nuclear magnetic resonance may be configured for enhancing a medicinal effect of a medication administered in the user's body. Further, the apparatus may include a controlling unit communicatively coupled with each of the magnetic unit and the radiofrequency coil unit. Further, the controlling unit may be configured for controlling one or more of the magnetic field characteristic and the radiofrequency characteristic to create a therapeutic effect in the user's body. Further, the therapeutic effect includes the medicinal effect.

The present disclosure provides an apparatus for inducing a nuclear magnetic resonance. Further, the apparatus may include a magnetic unit which may be configured for generating a static magnetic field. Further, the static magnetic field may be characterized by a magnetic field characteristic. Further, the apparatus may include a radiofrequency coil unit which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the apparatus may include a controlling unit communicatively coupled with each of the magnetic unit and the radiofrequency coil unit. Further, the controlling unit may be configured for controlling one or more of the magnetic field characteristic and the radiofrequency characteristic to create a therapeutic effect in the user's body. Further, the apparatus may include an input device which may be configured for receiving a requirement data. Further, the requirement data indicates the therapeutic effect required by a user. Further, the controlling unit may be communicatively coupled with the input device. Further, the controlling unit may be further configured for determining one or more of the magnetic field characteristic and the radiofrequency characteristic based on the requirement data. Further, the controlling of one or more of the magnetic field characteristic and the radiofrequency characteristic may be further based on the determining of one or more of the magnetic field characteristic and the radiofrequency characteristic.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 illustrates an apparatus 100 for inducing a nuclear magnetic resonance, in accordance with some embodiments.

FIG. 2 illustrates the apparatus 100 for inducing the nuclear magnetic resonance, in accordance with some embodiments.

FIG. 3 illustrates the apparatus 100 for inducing the nuclear magnetic resonance, in accordance with some embodiments.

FIG. 4 illustrates a block diagram of the apparatus 100 for inducing the nuclear magnetic resonance, in accordance with some embodiments.

FIG. 5 illustrates an apparatus 500 for inducing a nuclear magnetic resonance, in accordance with some embodiments.

FIG. 6 illustrates a nanoparticle 600 administered in a user's body, in accordance with some embodiments.

FIG. 7 illustrates an apparatus 700 for inducing a nuclear magnetic resonance, in accordance with some embodiments.

FIG. 8 illustrates a transducer 502 of the apparatus 500 for inducing a nuclear magnetic resonance, in accordance with some embodiments.

FIG. 9 illustrates a clinical application of the apparatus 500, in accordance with some embodiments.

FIG. 10 illustrates a micellar-nanoparticle 1000 administered in the user's body, in accordance with some embodiments.

FIG. 11 is an illustration of an online platform 1100 consistent with various embodiments of the present disclosure.

FIG. 12 is a block diagram of a computing device 1200 for implementing the methods disclosed herein, in accordance with some embodiments.

FIG. 13 illustrates a block diagram of an apparatus 1300 for inducing a nuclear magnetic resonance, in accordance with some embodiments.

FIG. 14 illustrates a block diagram of an apparatus 1400 for inducing a nuclear magnetic resonance, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive.

Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list. ”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes a Nuclear Magnetic Resonance (NMR) Molecular Transducer, comprised of a Nuclear Magnetic Resonance (NMR) Transducer which may be programmed to generate various known NMR signals for various generic medications. The NMR Transducer may use currently available technology to generate unique NMR signals either by directing a particular radio signal through a constant magnetic field to generate the unique NMR signal, or use of a computer-generated NMR Signal to generate the unique NMR signal for the particular medication.

Nuclear Magnetic Resonance (NMR) technology has been widely used in medical imaging and diagnostics for many years. Traditionally, NMR has been employed to obtain detailed images of internal body structures through the application of magnetic fields and radiofrequency signals. However, the use of NMR for therapeutic purposes remains largely unexplored.

Several existing technologies and devices utilize NMR and ultrasound transducers for various medical applications, including imaging and treatment. For example, some devices employ NMR transducers for imaging, while others use ultrasound for the ablation of lesions or high-frequency vibrations for targeted cell destruction. Despite these advancements, there has been no significant development in using NMR technology to deliver specific molecular signals to impart therapeutic effects of medications directly into the body of a patient.

The present disclosure describes the NMR Molecular Transducer capable of generating and delivering unique NMR signals corresponding to specific medications. The NMR Molecular Transducer has the potential to revolutionize the way medications are administered, offering a non-invasive, efficient, and customizable approach to patient care.

A Computer Processing Unit (CPU) would have to be programmed and developed such that the particular signal could be transmitted at a certain Amplitude or Intensity for a defined period of time to cause a predetermined effect based upon the weight of the patient. All of the patient's medications and known NMR signals would have to be stored in the computer's memory. Further, storing the patient's medications and known NMR signals would allow all medications to be instilled by the transducer via a transducer gel into the abdomen of the subject over a short-defined period of time. Both Animal and Human testing would have to be completed to determine the strength [Amplitude] of the NMR signal and the length of time required to cause the desired physiological effect of the medication and persist for a particular period of time.

If the signal was administered at home by the patient, administration could be done daily or weekly. But if the signal was administered by a nurse at a community clinic, possibly in the Third World or lower-income facility, the effect has to persist at a stable therapeutic level for at least 2-4 weeks. To maintain the stable therapeutic level, there is a need for a design of a biologically inert nanoparticle that would resonate at a particular NMR frequency within the body of the subject, stimulating affiliated water molecules to resonate at the same frequency over a prolonged period of time. The effect of the stimulation of the water molecules should cascade throughout the body to maintain a consistent signal throughout the body for a set period of time. Each patient's list of medications might be stored in the Computer Processing Unit (CPU) and might be administered to the patient in sequential order during a single session.

In a Community Clinic utilizing the device, the entire community of patients might be stored in the CPU, but a failsafe program has to be written to ensure that only a single individual receives the single individual's medication molecular profile. Biometrics, like a Thumb print recognition, may be a definitive method to ensure appropriate administration of medications to the correct patient only after a specified period of time. Interestingly, once all of the NMR signals for the particular device are recorded, the NMR signal might be transmitted via computer signals to other computer systems or by radio signals to other areas of the world, the moon, planets, or beyond in order to maintain a fully therapeutic pharmacy. This may allow remote medical units to always maintain a full pharmacy for the medical unit's patient needs, simply by ordering the required medication by radio or computer transmission.

Once all of the NMR signals for the particular device are recorded, the NMR signal could be transmitted via computer signals to other computer systems or by radio signals to other areas of the world, the moon, planets, or beyond in order to maintain a fully therapeutic pharmacy. This would allow remote medical units to always maintain a full pharmacy for the medical unit's patient needs, simply by ordering the required medication by radio or computer transmission.

In consideration of the prior art, many utilize an NMR Transducer for NMR Imaging, but not therapeutically. A few of the US devices are used therapeutically, but in a completely different manner. One Ultrasound device was strapped to the head for US treatment of intracerebral lesions. Another US probe compatible with MRI was used to visualize the lesion during US treatment to more accurately treat or destroy the lesion with US while monitoring its status by MRI to make it more accurate. Another medical device uses a high frequency vibration between 10-to 1000 hertz (Hz) for a period greater than 1 hour for ablation of a group of cells within the body. Finally, a US transducer was noted with concurrent voltage across the same area of the body to ablate lesions with greater efficiency. None of the above-mentioned prior art describes an NMR Transducer device designed to provide specific molecular signals to impart the effect of a particular medication into the body of a patient for ongoing therapeutic effects.

Transcranial Magnetic Stimulation (TMS) does use NMR to treat severe Depression or Anxiety which are not responsive to medications with significant but variable degrees of success. TMS utilizes a broad spectrum of NMR from 10,000 to 50,000 Hz transmitted across the skull and through the brain to stimulate certain areas of the brain, and thereby completely or partially relieve their mental health symptoms for a few months or up to a year before requiring another treatment. This device is similar to outmoded ECT[Electro-Convulsive Therapy] which involves passing a controlled electric current through the brain to induce a brief seizure. But, TMS uses a broad spectrum NMR signal and does not usually cause any seizure activity

If a Patented Medication was required for treatment, financial arrangements might be made with the particular pharmaceutical company for the use of the patented medication by an NMR Molecular Transducer for each patient application. And, the Pharmaceutical companies might benefit from no longer needing to mass produce large quantities of the medication for worldwide distribution, which might save immense amounts of manufacturing and production costs for the pharmaceutical companies'product.

Further, the NMR molecular transducer may utilize Nuclear Magnetic Resonance (NMR) signals tailored to the molecular structure of specific medications. Further, the NMR molecular transducer may allow non-invasive administration of medications by transmitting precisely programmed NMR signals into the body via a transducer, thereby stimulating molecular resonance associated with therapeutic compounds.

Further, the NMR molecular transducer may be coupled with a CPU programmable with a database of known NMR signal profiles corresponding to various medications.

The technical problem of inefficient and non-customizable drug administration may be solved by enabling signal generation based on patient-specific parameters such as weight, metabolism, and dosage schedule. Further, the CPU may modulate the amplitude, intensity, and duration of signals to achieve pharmacokinetically appropriate responses. For example, a signal intended to mimic ibuprofen may be delivered for 5 minutes at a 50 mT/m gradient with a radiofrequency pulse sequence matching the mimic ibuprofen's unique resonance signature.

Further, the biologically inert polar nanoparticles may be administered into the body to prolong and stabilize the resonance effect of the NMR signals. These nanoparticles may resonate at specific frequencies and may stimulate surrounding water molecules to maintain a cascading signal within tissues. The improvement may be in the field of resonance-enhanced drug retention, where existing technologies do not provide long-term molecular stimulation from external signal sources.

Further, a gel-based transducer may facilitate the delivery of NMR signals by enhancing signal conductivity across the epidermis and subcutaneous layers. This transdermal coupling may improve therapeutic penetration of the NMR field, solving the technical limitation of signal attenuation in biological tissues. For instance, an abdominal gel patch may be applied to direct the signal to hepatic pathways for drugs metabolized in the liver.

Further, the CPU may store and sequentially deliver multiple medication profiles for a single patient during one treatment session. This enhancement may address the technical limitation of polypharmacy management in remote or resource-constrained clinical environments, improving the technology of medication scheduling and compliance automation.

Further, biometric safeguards such as thumbprint recognition may be integrated with the CPU to ensure patient-specific signal administration, thereby solving the problem of inadvertent signal mismatch in shared community clinics. This improves the technology of secure therapeutic access control.

Further, the system may allow the transmission of NMR signal profiles via radio or computer signals to remote sites, including planetary or extraterrestrial medical stations. This feature may significantly improve telepharmacy and space medicine technologies, allowing real-time access to therapeutic signals without physical drug stockpiling.

Further, the molecular NMR signal transmitted into the body by Transducer, wireless Transmitter Pen, or gelatin based bandage with embedded Receiver/Transmitter may be controlled by the users personal cell phone or a mobile Clinic Based Computer to generate the NMR signal for transmission into the body to cause a specific NMR molecular signal within the body which would lead to a specific molecular therapeutic effect of the medication. This NMR signal may be a recorded signal which has been generated in a laboratory or commercial business which simply maintains a library of molecular NMR signals. The NMR molecular signal recordings can be sent by radio signal, email, or via cell phone to a specific receiver discussed above and then transmitted by Transducer, Pen Transmitter, Bandage-gel transmitter, or direct transmission into the body to cause the therapeutic effect of the medication.

Further, AI-enhanced adaptive signal modulation may be introduced wherein the CPU utilizes real-time biosensor feedback (e.g., heart rate, hydration, glucose level) to dynamically adjust NMR signal amplitude and duration. This may solve the problem of static dosing in fluctuating physiological states. For instance, a diabetic patient's glucose monitor may prompt the transducer to increase the therapeutic insulin-mimicking signal in response to a detected spike.

Further, quantum dot-based signal enhancers may be incorporated within the nanoparticle matrix to increase NMR signal sharpness and target specificity. The quantum dots may be engineered to emit secondary magnetic responses, solving the technical limitation of signal dispersion and increasing localization for specific tissue targets like cancerous cells.

Further, machine-learned molecular signal synthesis may be used, wherein the CPU is trained on large datasets of NMR spectra and drug responses to generate synthetic resonance profiles for medications whose native signatures are unknown or unstable. This may enhance automated pharmacogenomic adaptation for personalized medicine. For example, a new peptide drug lacking NMR data may have its signal inferred using generative adversarial networks trained on structural analogs.

Further, the CPU may employ hydrogel-based smart patches containing embedded sensors and micro-antennas to detect signal absorption efficacy and tissue penetration levels. These hydrogels may change their dielectric properties in response to resonance activity, allowing the CPU to fine-tune the transducer's orientation and power delivery. This improvement may solve the technical problem of variability in transdermal signal consistency.

Further, multi-modal signal synchronization may be implemented wherein NMR signals are combined with mild electrical or ultrasound stimulation to amplify therapeutic effects. For instance, synchronizing a low-voltage electric field with the resonance window of a neurotransmitter mimic may enhance synaptic uptake in neurodegenerative treatments, thereby improving the technology of synergistic bio-signal therapy.

In some embodiments, the apparatus for inducing the nuclear magnetic resonance may include a portable housing. Further, the portable housing may be configured to facilitate therapeutic use in one or more of an ambulatory setting and a remote setting. Further, the apparatus may include a magnetic unit configured for generating a static magnetic field. Further, the magnetic unit is characterized by a reduced form factor and utilizes one or more of a permanent magnet and a miniaturized superconducting magnet.

In some embodiments, the apparatus may further include a radiofrequency (RF) coil unit configured for generating a radiofrequency pulse. Further, the radiofrequency coil unit may be embedded within a flexible pad or wearable patch configured to be applied to a skin surface of a user. Further, the radiofrequency coil unit may be communicatively coupled to a control unit. Further, the control unit may be configured for controlling one or more of a magnetic field characteristic and a radiofrequency characteristic to induce the nuclear magnetic resonance in the user's body.

In some embodiments, the control unit may be powered by a battery module and may include a memory configured for storing one or more signal profiles corresponding to a plurality of medications. Further, the control unit may include a processing module configured for selecting a signal profile based on one or more of a requirement data, a medication data, and a physiological parameter of the user.

In some embodiments, the apparatus may include a biometric authentication module configured for authenticating a user identity using biometric data, such as a thumbprint, prior to the inducing of the nuclear magnetic resonance.

In some embodiments, the apparatus may include a wireless communication module configured for receiving therapeutic profiles or transmitting session data to a remote server.

In some embodiments, the apparatus for inducing the nuclear magnetic resonance comprises a retractable pin array configured for delivering a radiofrequency pulse sub-dermally into the user's body. Further, the apparatus may include a magnetic unit configured for generating a static magnetic field and a radiofrequency coil unit configured for generating a radiofrequency pulse. Further, the radiofrequency coil unit is communicatively coupled to a pin delivery module.

In some embodiments, a pin delivery module comprises a plurality of electrically conductive and retractable pins. Further, the pins are configured to extend into a tissue layer of the user during operation and retract upon session termination. Further, the pins may be composed of a biocompatible conductive material and may be actuated using an electromechanical actuator under the control of a control unit. Further, the electrochemical actuator may include a solenoid and a piezoelectric driver.

In some embodiments, the radiofrequency coil unit may be configured for electrically coupling with the retractable pin array, such that the radiofrequency pulse is transmitted through the pins to induce the nuclear magnetic resonance within subcutaneous or intramuscular tissues.

In some embodiments, the control unit may be further configured to modulate amplitude, phase, and duration of the radiofrequency pulse based on a depth of pin penetration and at least one physiological parameter of the user.

In some embodiments, the retractable pins include at least one of impedance sensors and temperature sensors configured for monitoring tissue coupling and thermal safety during operation.

In some embodiments, the retractable pins may be hollow and comprise longitudinal grooves. Further, the retractable pins are configured for delivering conductive media, nanoparticle suspensions, and a therapeutic gel to the tissue during pin deployment.

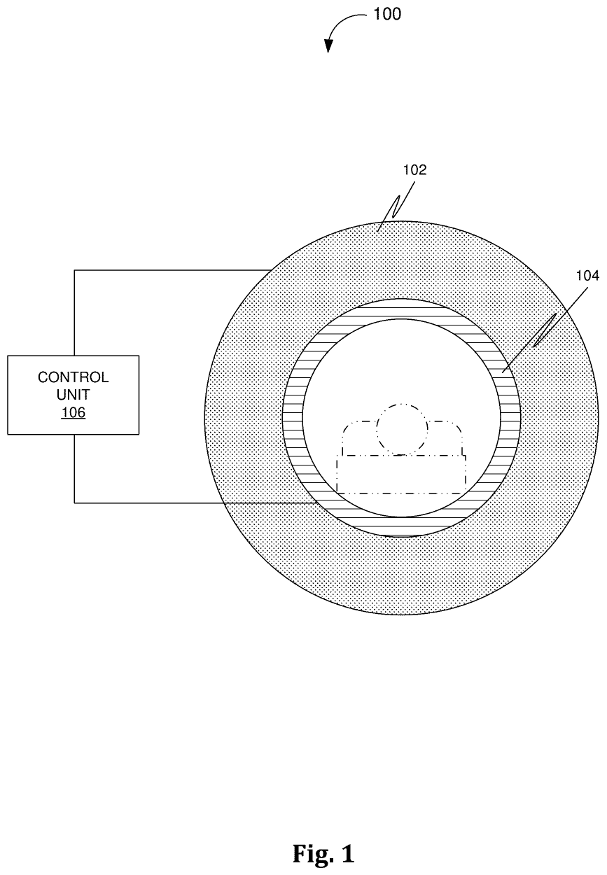

FIG. 1 illustrates an apparatus 100 for inducing a nuclear magnetic resonance, in accordance with some embodiments.

Accordingly, the apparatus 100 may include a magnetic unit 102 which may be configured for generating a static magnetic field. Further, the static magnetic field may be characterized by a magnetic field characteristic. Further, the apparatus 100 may include a radiofrequency coil unit 104 which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the apparatus may include a controlling unit 106 communicatively coupled with each of the magnetic unit 102 and the radiofrequency coil unit 104. Further, the controlling unit may be configured for controlling one or more of the magnetic field characteristic and the radiofrequency characteristic to create a therapeutic effect in the user's body.

In some embodiments, the therapeutic effect corresponds to one or more of a physiological response of the user's body to the nuclear magnetic resonance and a psychological response of a user to the nuclear magnetic resonance.

In some embodiments, the nuclear magnetic resonance may be configured for enhancing a medicinal effect of a medication administered in the user's body. Further, the therapeutic effect includes the medicinal effect.

In some embodiments, the nuclear magnetic resonance may be further configured for inducing a biological process in the user's body. Further, the enhancing of the medicinal effect may be based on the inducing of the biological process. Further, the medication may be configured to provide the medicinal effect using the biological process.

In some embodiments, the medication may be administered in the user's body at a time instance (i.e., time instant). Further, the time instance occurs one or more of before inducing the nuclear magnetic resonance, after the inducing of the nuclear magnetic resonance, and during the inducing of the nuclear magnetic resonance.

In some embodiments, the controlling of one or more of the magnetic field characteristic and the radiofrequency characteristic includes generating one or more of a magnetic field-control signal and a radiofrequency-control signal. Further, the magnetic unit 102 may be further configured for receiving the magnetic field-control signal.

Further, the generating of the static magnetic field may be based on the magnetic field-control signal. Further, the radiofrequency coil unit 104 may be further configured for receiving the radiofrequency-control signal. Further, the generating of the radiofrequency pulse may be based on the radiofrequency-control signal.

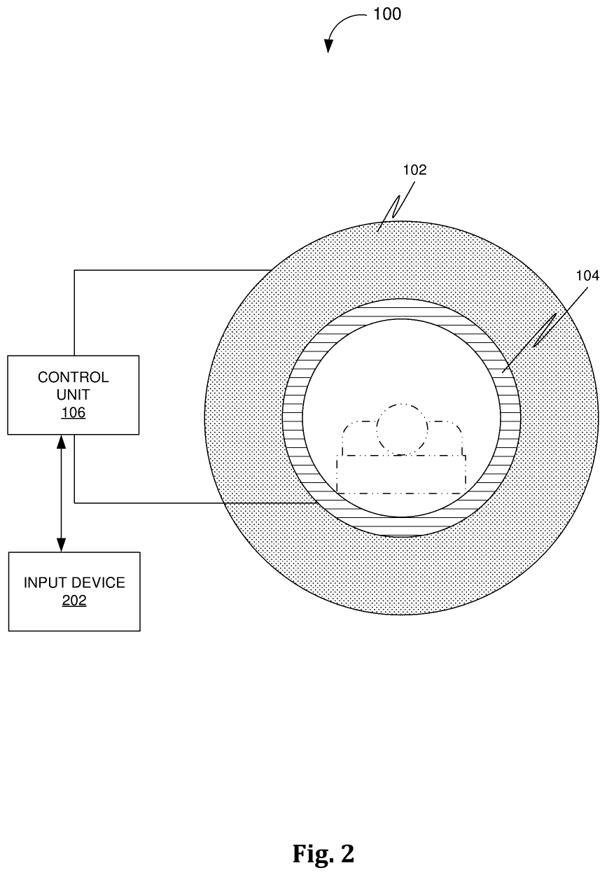

FIG. 2 illustrates the apparatus 100 for inducing the nuclear magnetic resonance, in accordance with some embodiments.

In some embodiments, the apparatus 100 may further include an input device 202 which may be configured for receiving a requirement data. Further, the requirement data indicates the therapeutic effect required by a user. Further, the controlling unit 106 may be communicatively coupled with the input device. Further, the controlling unit may be further configured for determining one or more of the magnetic field characteristic and the radiofrequency characteristic based on the requirement data. Further, the generating of one or more of the magnetic field-control signal and the radiofrequency-control signal may be further based on the determining of one or more of the magnetic field characteristic and the radiofrequency characteristic.

In some embodiments, one or more of the static magnetic field and the radiofrequency pulse may be configured to induce the nuclear magnetic resonance of a nanoparticle administered in the user's body. Further, the nuclear magnetic resonance of the nanoparticle stimulates an atomic nucleus of a molecule to undergo the nuclear magnetic resonance in the user's body. Further, the molecule may be comprised in the user's body. Further, the nuclear magnetic resonance of the atomic nucleus creates the therapeutic effect in the user's body.

In some embodiments, the nuclear magnetic resonance of the nanoparticle stimulates the atomic nucleus to undergo the nuclear magnetic resonance in the user's body over a time interval. Further, the nuclear magnetic resonance of the atomic nucleus over the time interval creates the therapeutic effect in the user's body.

In some embodiments, the nuclear magnetic resonance of the atomic nucleus of the molecule stimulates the atomic nucleus of two or more molecules to undergo the nuclear magnetic resonance in the user's body. Further, the nuclear magnetic resonance of the two or more molecules creates the therapeutic effect in the user's body.

In some embodiments, the molecule includes a water molecule.

In some embodiments, the nanoparticle may be characterized by a bio-inertness property.

In some embodiments, the input device 202 may be further configured for receiving a physiological parameter of the user's body. Further, the determining of one or more of the magnetic field characteristic and the radiofrequency characteristic based on the requirement data may be further based on the physiological parameter of the user's body.

In some embodiments, the input device 202 may be further configured for receiving a medication data. Further, the medication data indicates a medication administered to the user's body. Further, the determining of one or more of the magnetic field characteristic and the radiofrequency characteristic may be further based on the medication data. Further, nuclear magnetic resonance may be configured for inducing a biological process in the user's body to enhance a medicinal effect of the medication. Further, the therapeutic effect includes the medicinal effect.

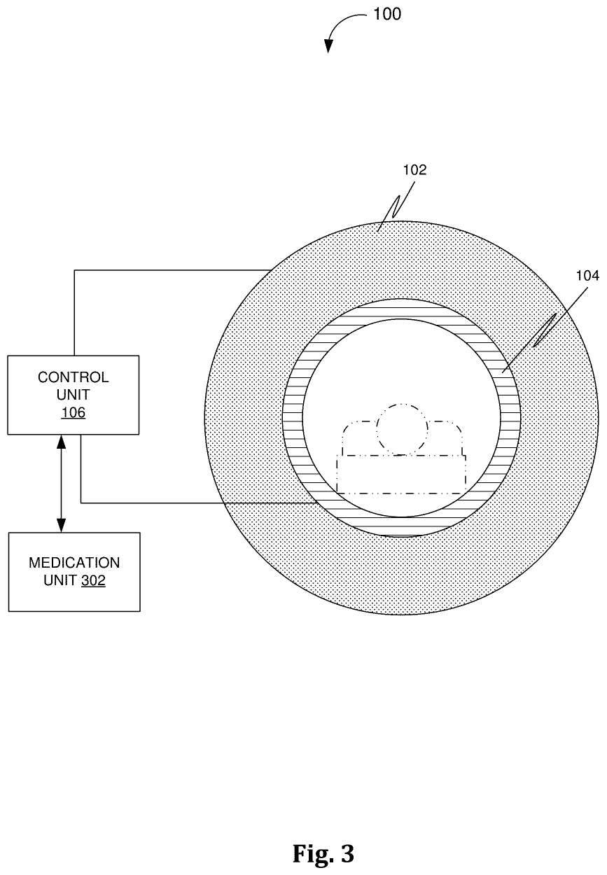

FIG. 3 illustrates the apparatus 100 for inducing the nuclear magnetic resonance, in accordance with some embodiments.

In some embodiments, the apparatus 100 may further include a medication unit 302 which may be configured for administering a medication to the user's body through a conductive medium. Further, the conductive medium may be applied to a skin of the user's body. Further, the conductive medium may be configured to facilitate the administering of the medication. Further, the controlling unit 106 may be communicatively coupled with the medication unit 302. Further, the controlling unit 106 may be further configured for controlling the administering of the medication. Further, the nuclear magnetic resonance may be configured for enhancing a medicinal effect of the medication. Further, the therapeutic effect includes the medicinal effect.

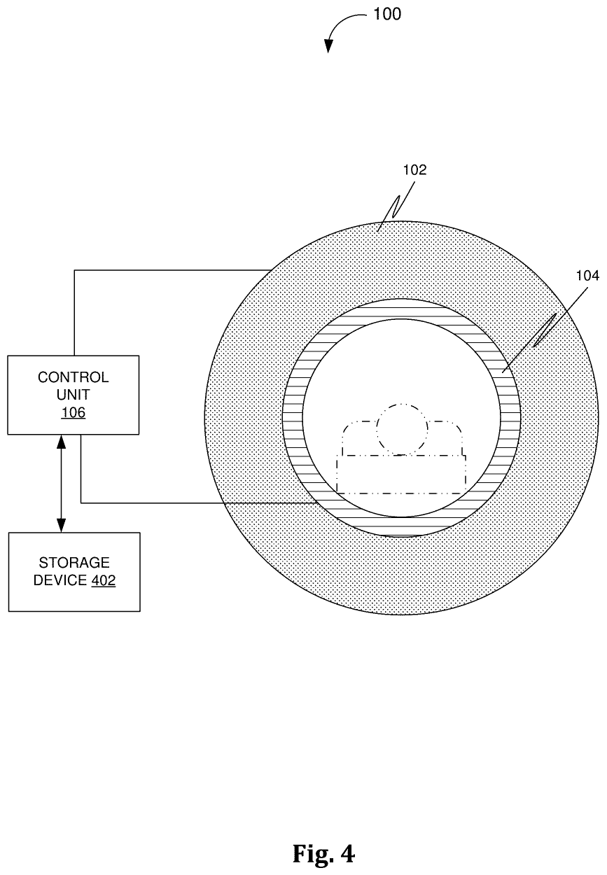

FIG. 4 illustrates a block diagram of the apparatus 100 for inducing the nuclear magnetic resonance, in accordance with some embodiments.

In some embodiments, the controlling unit 106 may be further configured for generating a user data. Further, the user data includes a detail associated with the inducing of the nuclear magnetic resonance in the user's body. Further, the apparatus 100 further includes a storage device 402 which may be configured for storing the user data in a database.

In some embodiments, the magnetic field characteristic includes a magnetic field strength.

In some embodiments, the radiofrequency characteristic includes a frequency of the radiofrequency pulse, a width of the radiofrequency pulse, a shape of the radiofrequency pulse, an amplitude of the radiofrequency pulse, and a phase of the radiofrequency pulse.

In some embodiments, the physiological parameter includes a weight of the user's body.

In some embodiments, the detail indicates one or more of the magnetic field characteristic, the radiofrequency characteristic, the therapeutic effect, and a time duration of the nuclear magnetic resonance.

In some embodiments, the user data further indicates a medication administered in the user's body.

In some embodiments, the user's body includes an animal's body and a human's body.

In some embodiments, the time interval ranges from two to four weeks.

In some embodiments, the database further includes a plurality of user data comprising details associated with the inducing of the nuclear magnetic resonance in the plurality of user's bodies.

In some embodiments, the apparatus 100 may further include an input device 202, as shown in FIG. 2, which may be configured for receiving a biometric data of the user. Further, the biometric data includes a biometric of the user. Further, the controlling unit 106 may be communicatively coupled with the input device 202. Further, the controlling unit 106 may be configured for analyzing the biometric data. Further, the controlling of the administrating of the medication may be further based on the analyzing of the biometric data.

In some embodiments, the controlling unit 106 may be further configured for determining a medication parameter of the user based on the analyzing of the biometric data. Further, the controlling of the administrating of the medication may be further based on the determining of the medication parameter.

In some embodiments, the medication parameter includes one or more of a quantity of the medication and a time interval between two or more medication administrations.

In some embodiments, the conductive medium includes a transducer gel.

In some embodiments, the biometric data includes a thumbprint of the user.

In some embodiments, the magnetic unit 102 includes a superconducting magnet.

In some embodiments, the radiofrequency coil unit 104 includes a radiofrequency coil.

In some embodiments, the nanoparticle includes a polymeric nanoparticle.

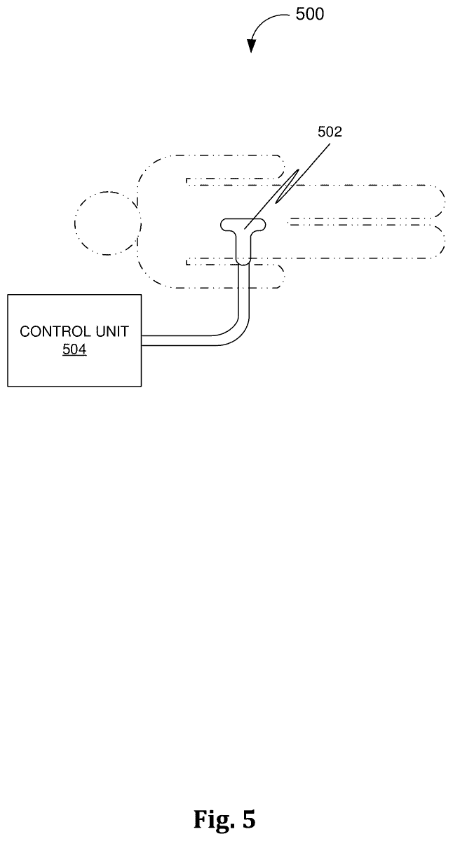

FIG. 5 illustrates an apparatus 500 for inducing a nuclear magnetic resonance, in accordance with some embodiments.

In some embodiments, the apparatus 500 may include a transducer 502 and a control unit 504. Further, the control unit 504 may be communicatively coupled with the transducer 502. Further, the transducer 502 may be configured for performing one or more operations for inducing the nuclear magnetic resonance in a user's body. Further, the control unit 504 may be configured for controlling the one or more operations of the transducer 502 to create a therapeutic effect in the user's body.

In some embodiments, the one or more operations may include directing a radiofrequency pulse through a static magnetic field.

In some embodiments, the transducer 502 may include a nuclear magnetic resonance (NMR) transducer.

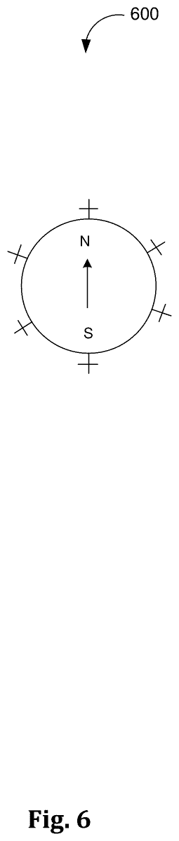

FIG. 6 illustrates a nanoparticle 600 administered in a user's body, in accordance with some embodiments.

In some embodiments, the nuclear magnetic resonance may be configured to induce the nuclear magnetic resonance of the nanoparticle 600 administered to the user's body. Further, the nuclear magnetic resonance of the nanoparticle 600 may induce the nuclear magnetic resonance of an atomic nucleus of a molecule to create the therapeutic effect. Further, the user's body comprises the molecule.

In some embodiments, the nanoparticle 600 may include a polar nanoparticle.

In some embodiments, the nanoparticle 600 may be characterized by a bio-inertness property.

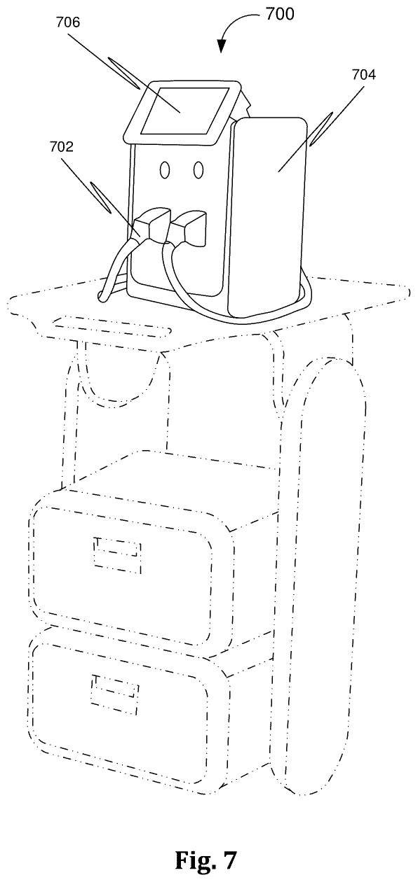

FIG. 7 illustrates an apparatus 700 for inducing a nuclear magnetic resonance, in accordance with some embodiments.

In some embodiments, the apparatus 700 may include a transducer probe 702 which may be configured for performing one or more operations for inducing the nuclear magnetic resonance in a user's body. Further, the apparatus 700 may include a controlling unit 704 communicatively coupled with the transducer probe 702. Further, the controlling unit 704 may be configured for controlling the one or more operations of the transducer 502 to create a therapeutic effect in the user's body.

In some embodiments, the apparatus 700 may include a presentation device 706 communicatively coupled with the controlling unit 704. Further, the presentation device 706 may be configured for presenting an operational parameter data. Further, the operational parameter data may include the operational parameter associated with the inducing of the nuclear magnetic resonance. Further, the controlling unit 704 may be configured for generating the operational parameter data.

In some embodiments, the transducer probe 702 may include two or more transducer probes.

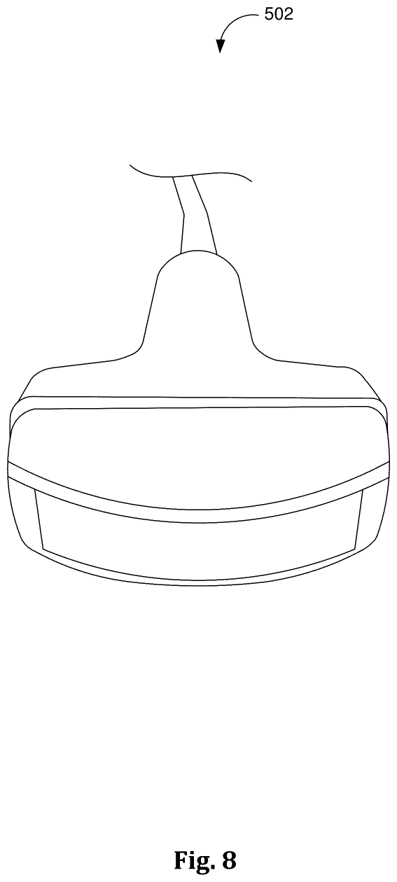

FIG. 8 illustrates a transducer 502 of the apparatus 500 for inducing a nuclear magnetic resonance, in accordance with some embodiments.

In some embodiments, the transducer 502 may include a transducer probe.

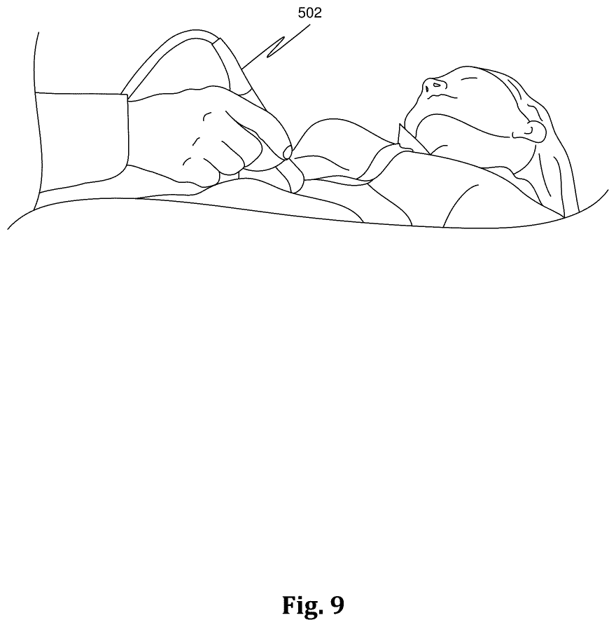

FIG. 9 illustrates a clinical application of the apparatus 500, in accordance with some embodiments.

In some embodiments, the user may be in a supine position during the clinical application of the apparatus 500. Further, the transducer 502 may be placed over an abdominal region of the user's body to induce the nuclear magnetic resonance.

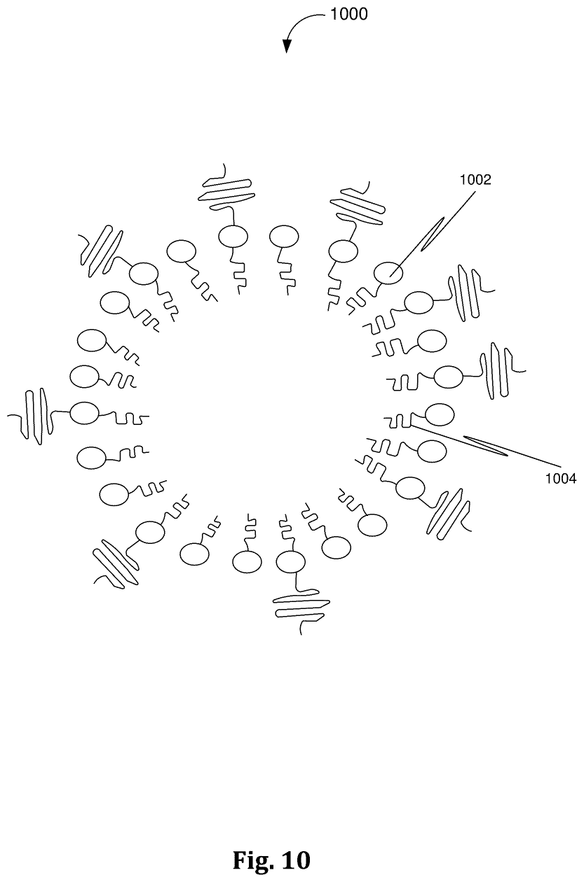

FIG. 10 illustrates a micellar-nanoparticle 1000 administered in the user's body, in accordance with some embodiments.

In some embodiments, the nanoparticle may include the micellar-nanoparticle 1000. Further, the micellar-nanoparticle 1000 may include two or more hydrophilic heads 1002 and two or more hydrophobic tails 1004.

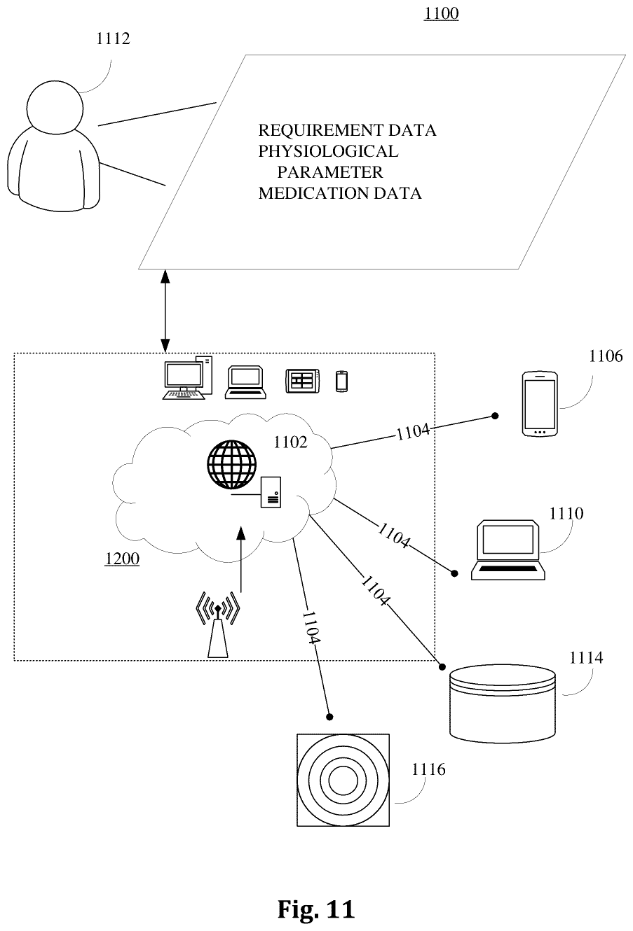

FIG. 11 is an illustration of an online platform 1100 consistent with various embodiments of the present disclosure. By way of non-limiting example, the online platform 1100 may be hosted on a centralized server 1102, such as, for example, a cloud computing service. The centralized server 1102 may communicate with other network entities, such as, for example, a mobile device 1106 (such as a smartphone, a laptop, a tablet computer etc.), other electronic devices 1110 (such as desktop computers, server computers etc.), databases 1114, and sensors 1116 over a communication network 1104, such as, but not limited to, the Internet. Further, users of the online platform 1100 may include relevant parties such as, but not limited to, end-users, administrators, service providers, service consumers and so on. Accordingly, in some instances, electronic devices operated by the one or more relevant parties may be in communication with the platform.

A user 1112, such as the one or more relevant parties, may access online platform 1100 through a web based software application or browser. The web based software application may be embodied as, for example, but not be limited to, a website, a web application, a desktop application, and a mobile application compatible with a computing device 1200.

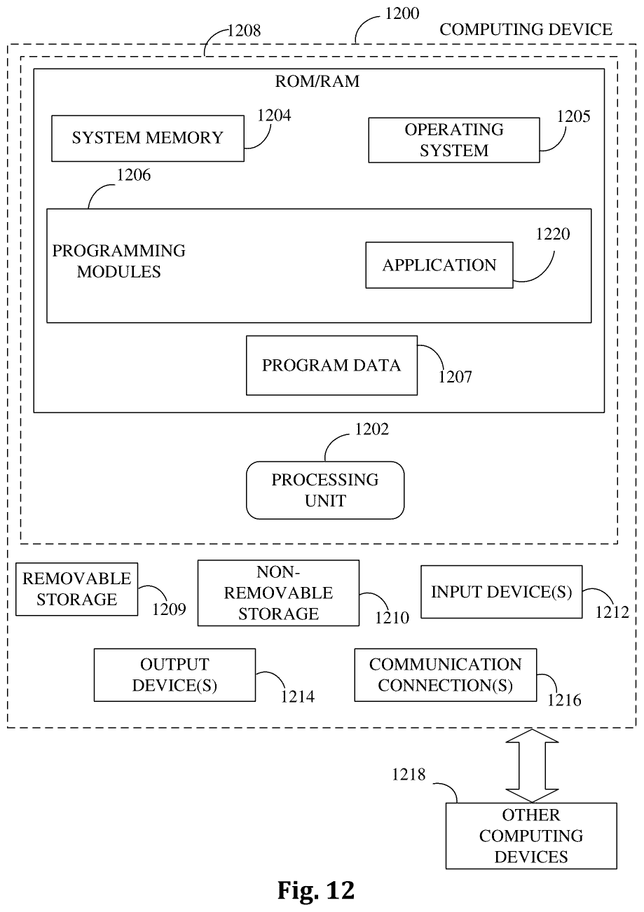

With reference to FIG. 12, a system consistent with an embodiment of the disclosure may include a computing device or cloud service, such as computing device 1200. In a basic configuration, computing device 1200 may include at least one processing unit 1202 and a system memory 1204. Depending on the configuration and type of computing device, system memory 1204 may comprise, but is not limited to, volatile (e.g. random-access memory (RAM)), non-volatile (e.g. read-only memory (ROM)), flash memory, or any combination. System memory 1204 may include operating system 1205, one or more programming modules 1206, and may include a program data 1207. Operating system 1205, for example, may be suitable for controlling computing device 1200's operation. In one embodiment, programming modules 1206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 12 by those components within a dashed line 1208.

Computing device 1200 may have additional features or functionality. For example, computing device 1200 may also include additional data storage devices (removable and/or non-removable) such as, for example, magnetic disks, optical disks, or tape. Such additional storage is illustrated in FIG. 12 by a removable storage 1209 and a non-removable storage 1210. Computer storage media may include volatile and non-volatile, removable and non-removable media implemented in any method or technology for storage of information, such as computer-readable instructions, data structures, program modules, or other data. System memory 1204, removable storage 1209, and non-removable storage 1210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1200. Any such computer storage media may be part of device 1200. Computing device 1200 may also have input device(s) 1212 such as a keyboard, a mouse, a pen, a sound input device, a touch input device, a location sensor, a camera, a biometric sensor, etc. Output device(s) 1214 such as a display, speakers, a printer, etc. may also be included. The aforementioned devices are examples and others may be used.

Computing device 1200 may also contain a communication connection 1216 that may allow device 1200 to communicate with other computing devices 1218, such as over a network in a distributed computing environment, for example, an intranet or the Internet. Communication connection 1216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1204, including operating system 1205. While executing on processing unit 1202, programming modules 1206 (e.g., application 1220 such as a media player) may perform processes including, for example, one or more stages of methods, algorithms, systems, applications, servers, databases as described above. The aforementioned process is an example, and processing unit 1202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

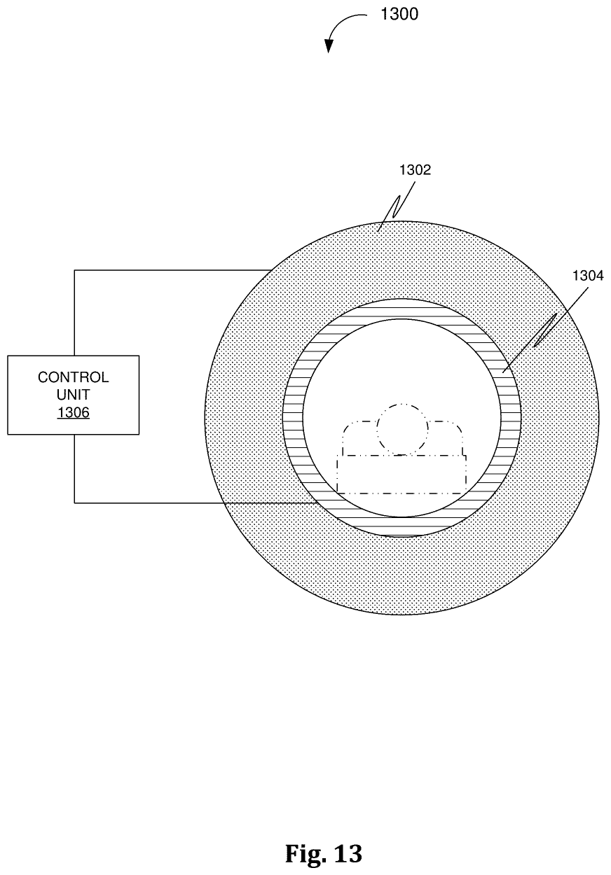

FIG. 13 illustrates a block diagram of an apparatus 1300 for inducing a nuclear magnetic resonance, in accordance with some embodiments.

Accordingly, the apparatus 1300 may include a magnetic unit 1302 which may be configured for generating a static magnetic field. Further, the static magnetic field may be characterized by a magnetic field characteristic. Further, the apparatus 1300 may include a radiofrequency coil unit 1304 which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the nuclear magnetic resonance may be configured for enhancing a medicinal effect of a medication administered in the user's body. Further, the apparatus 1300 may include a controlling unit 1306 communicatively coupled with each of the magnetic unit 1302 and the radiofrequency coil unit 1304. Further, the controlling unit 1306 may be configured for controlling one or more of the magnetic field characteristic and the radiofrequency characteristic to create a therapeutic effect in the user's body. Further, the therapeutic effect includes the medicinal effect.

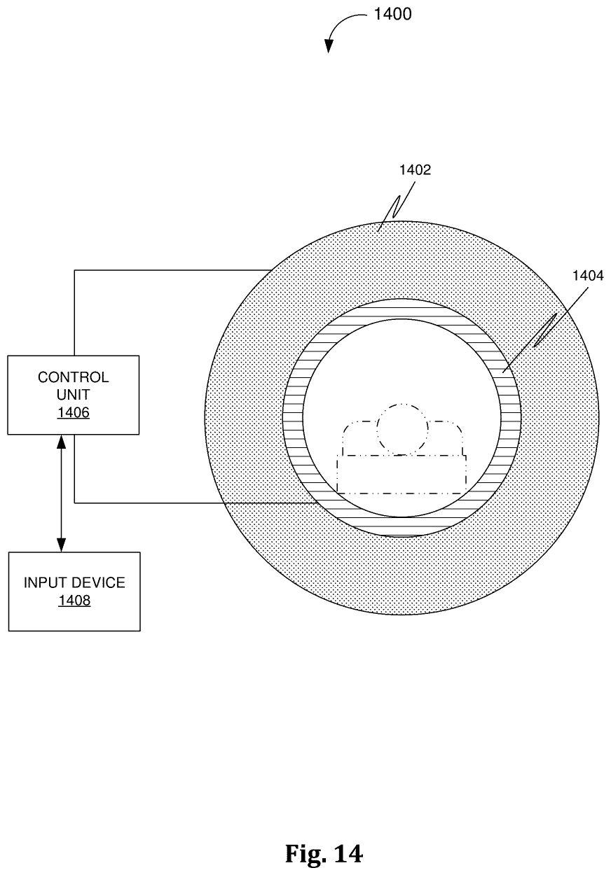

FIG. 14 illustrates a block diagram of an apparatus 1400 for inducing a nuclear magnetic resonance, in accordance with some embodiments.

Accordingly, the apparatus 1400 may include a magnetic unit 1402 which may be configured for generating a static magnetic field. Further, the static magnetic field may be characterized by a magnetic field characteristic. Further, the apparatus 1400 may include a radiofrequency coil unit 1404 which may be configured for generating a radiofrequency pulse. Further, the radiofrequency pulse may be characterized by a radiofrequency characteristic. Further, the static magnetic field and the radiofrequency pulse may be configured to induce the nuclear magnetic resonance in a user's body. Further, the apparatus 1400 may include a controlling unit 1406 communicatively coupled with each of the magnetic unit 1402 and the radiofrequency coil unit 1404. Further, the controlling unit 1406 may be configured for controlling one or more of the magnetic field characteristic and the radiofrequency characteristic to create a therapeutic effect in the user's body. Further, the apparatus 1400 may include an input device 1408 which may be configured for receiving a requirement data. Further, the input data indicates the therapeutic effect required by a user. Further, the controlling unit 1406 may be communicatively coupled with the input device 1408. Further, the controlling unit 1406 may be further configured for determining one or more of the magnetic field characteristic and the radiofrequency characteristic based on the requirement data. Further, the controlling of one or more of the magnetic field characteristic and the radiofrequency characteristic may be further based on the determining of one or more of the magnetic field characteristic and the radiofrequency characteristic.

Although the invention has been explained in relation to its preferred embodiment, it is to be understood that many other possible modifications and variations can be made without departing from the spirit and scope of the invention as hereinafter claimed.

What is claimed is:

1. An apparatus for inducing a nuclear magnetic resonance, the apparatus comprising:
a magnetic unit configured for generating a static magnetic field, wherein the static magnetic field is characterized by a magnetic field characteristic;
a radiofrequency coil unit configured for generating a radiofrequency pulse, wherein the radiofrequency pulse is characterized by a radiofrequency characteristic, wherein the static magnetic field and the radiofrequency pulse are configured to induce the nuclear magnetic resonance in a user's body; and
a controlling unit communicatively coupled with each of the magnetic unit and the radiofrequency coil unit, wherein the controlling unit is configured for controlling at least one of the magnetic field characteristic and the radiofrequency characteristic to create a therapeutic effect in the user's body.

2. The apparatus of claim 1, wherein the therapeutic effect corresponds to at least one of a physiological response of the user's body to the nuclear magnetic resonance and a psychological response of a user to the nuclear magnetic resonance.

3. The apparatus of claim 1, wherein the nuclear magnetic resonance is configured for enhancing a medicinal effect of a medication administered in the user's body, wherein the therapeutic effect comprises the medicinal effect.

4. The apparatus of claim 3, wherein the nuclear magnetic resonance is further configured for inducing a biological process in the user's body, wherein the enhancing of the medicinal effect is based on the inducing of the biological process, wherein the medication is configured to provide the medicinal effect using the biological process.

5. The apparatus of claim 3, wherein the medication is administered in the user's body at a time instance, wherein the time instance occurs at least one of before inducing the nuclear magnetic resonance, after the inducing of the nuclear magnetic resonance, and during the inducing of the nuclear magnetic resonance.

6. The apparatus of claim 1, wherein the controlling of at least one of the magnetic field characteristic and the radiofrequency characteristic comprises generating at least one of a magnetic field-control signal and a radiofrequency-control signal, wherein the magnetic unit is further configured for receiving the magnetic field-control signal, wherein the generating of the static magnetic field is based on the magnetic field-control signal, wherein the radiofrequency coil unit is further configured for receiving the radiofrequency-control signal, wherein the generating of the radiofrequency pulse is based on the radiofrequency-control signal.

7. The apparatus of claim 6 further comprising an input device configured for receiving a requirement data, wherein the requirement data indicates the therapeutic effect required by a user, wherein the controlling unit is communicatively coupled with the input device, wherein the controlling unit is further configured for determining at least one of the magnetic field characteristic and the radiofrequency characteristic based on the requirement data, wherein the generating of at least one of the magnetic field-control signal and the radiofrequency-control signal is further based on the determining of at least one of the magnetic field characteristic and the radiofrequency characteristic.

8. The apparatus of claim 1, wherein at least one of the static magnetic field and the radiofrequency pulse is configured to induce the nuclear magnetic resonance of a nanoparticle administered in the user's body, wherein the nuclear magnetic resonance of the nanoparticle stimulates an atomic nucleus of a molecule to undergo the nuclear magnetic resonance in the user's body, wherein the molecule is comprised in the user's body, wherein the nuclear magnetic resonance of the atomic nucleus creates the therapeutic effect in the user's body.

9. The apparatus of claim 8, wherein the nuclear magnetic resonance of the nanoparticle stimulates the atomic nucleus to undergo the nuclear magnetic resonance in the user's body over a time interval, wherein the nuclear magnetic resonance of the atomic nucleus over the time interval creates the therapeutic effect in the user's body.

10. The apparatus of claim 8, wherein the nuclear magnetic resonance of the atomic nucleus of the molecule stimulates the atomic nucleus of a plurality of molecules to undergo the nuclear magnetic resonance in the user's body, wherein the nuclear magnetic resonance of the plurality of molecules creates the therapeutic effect in the user's body.

11. The apparatus of claim 8, wherein the molecule comprises a water molecule.

12. The apparatus of claim 8, wherein the nanoparticle is characterized by a bio-inertness property.

13. The apparatus of claim 7, wherein the input device is further configured for receiving a physiological parameter of the user's body, wherein the determining of at least one of the magnetic field characteristic and the radiofrequency characteristic based on the requirement data is further based on the physiological parameter of the user's body.

14. The apparatus of claim 7, wherein the input device is further configured for receiving a medication data, wherein the medication data indicates a medication administered in the user's body, wherein the determining of at least one of the magnetic field characteristic and the radiofrequency characteristic is further based on the medication data, wherein the nuclear magnetic resonance is configured for inducing a biological process in the user's body to enhance a medicinal effect of the medication, wherein the therapeutic effect comprises the medicinal effect.

15. The apparatus of claim 1 further comprising a medication unit configured for administering a medication to the user's body through a conductive medium, wherein the conductive medium is applied on a skin of the user's body, wherein the conductive medium is configured to facilitate the administering of the medication, wherein the controlling unit is communicatively coupled with the medication unit, wherein the controlling unit is further configured for controlling the administering of the medication, wherein the nuclear magnetic resonance is configured for enhancing a medicinal effect of the medication, wherein the therapeutic effect comprises the medicinal effect.

16. The apparatus of claim 1, wherein the controlling unit is further configured for generating a user data, wherein the user data comprises a detail associated with the inducing of the nuclear magnetic resonance in the user's body, wherein the apparatus further comprises a storage device configured for storing the user data in a database.

17. The apparatus of claim 1, wherein the magnetic field characteristic comprises a magnetic field strength.

18. The apparatus of claim 1, wherein the radiofrequency characteristic comprises a frequency of the radiofrequency pulse, a width of the radiofrequency pulse, a shape of the radiofrequency pulse, an amplitude of the radiofrequency pulse, and a phase of the radiofrequency pulse.

19. An apparatus for inducing a nuclear magnetic resonance, the apparatus comprising:
a magnetic unit configured for generating a static magnetic field, wherein the static magnetic field is characterized by a magnetic field characteristic;
a radiofrequency coil unit configured for generating a radiofrequency pulse, wherein the radiofrequency pulse is characterized by a radiofrequency characteristic, wherein the static magnetic field and the radiofrequency pulse are configured to induce the nuclear magnetic resonance in a user's body, wherein the nuclear magnetic resonance is configured for enhancing a medicinal effect of a medication administered in the user's body; and
a controlling unit communicatively coupled with each of the magnetic unit and the radiofrequency coil unit, wherein the controlling unit is configured for controlling at least one of the magnetic field characteristic and the radiofrequency characteristic to create a therapeutic effect in the user's body, wherein the therapeutic effect comprises the medicinal effect.

20. An apparatus for inducing a nuclear magnetic resonance, the apparatus comprising:
a magnetic unit configured for generating a static magnetic field, wherein the static magnetic field is characterized by a magnetic field characteristic;
a radiofrequency coil unit configured for generating a radiofrequency pulse, wherein the radiofrequency pulse is characterized by a radiofrequency characteristic, wherein the static magnetic field and the radiofrequency pulse are configured to induce the nuclear magnetic resonance in a user's body;
a controlling unit communicatively coupled with each of the magnetic unit and the radiofrequency coil unit, wherein the controlling unit is configured for controlling at least one of the magnetic field characteristic and the radiofrequency characteristic to create a therapeutic effect in the user's body; and
an input device configured for receiving a requirement data, wherein the input data indicates the therapeutic effect required by a user, wherein the controlling unit is communicatively coupled with the input device, wherein the controlling unit is further configured for determining at least one of the magnetic field characteristic and the radiofrequency characteristic based on the requirement data, wherein the controlling of at least one of the magnetic field characteristic and the radiofrequency characteristic is further based on the determining of at least one of the magnetic field characteristic and the radiofrequency characteristic.

21. The molecular NMR signal transmitted into the body by Transducer, wireless Transmitter Pen, or gelatin based bandage with embedded Receiver/Transmitter which may be controlled by the users personal cell phone or a mobile Clinic Based Computer to generate the NMR signal for transmission into the body to cause a specific NMR molecular signal within the body which would lead to a specific molecular therapeutic effect of the medication. This NMR signal may be a recorded signal which has been generated in a laboratory or commercial business which maintains a library of molecular NMR signals. The NMR molecular signal recordings can be sent by radio signal, email, or via cell phone to a specific receiver discussed above and then transmitted by Transducer, Pen Transmitter, Bandage-gel transmitter, or direct transmission into the body to cause the therapeutic effect of the medication. In this application, there would be no need to generate the NMR signal at the bedside or at home to administer the NMR Molecular signal into the individual's body since the NMR signal has already been produced in the laboratory or commercial setting
